(19) United States
(12) Patent Application Publication
(10) Pub. No.: US 2022/0406466 A1
(43) Pub. Date: Dec. 22, 2022

(54) A METHOD FOR DETERMINING A RISK SCORE FOR A PATIENT

(71) Applicant: Mölnlycke Health Care AB, Göteborg (SE)

(72) Inventor: Brian Andrews, Göteborg (SE)

(21) Appl. No.: 17/781,196

(22) PCT Filed: Dec. 2, 2020
(86) PCT No.: PCT/SE2020/051160

§ 371 Date: May 31, 2022

(30) Foreign Application Priority Data
Dec. 3, 2019 (SE) 1951381-1

(51) Int. Cl. G16H 50/30; G16H 50/70; G16H 10/60

(52) U.S. Cl. CPC G16H 50/30; G16H 50/70; G16H 10/60

(57) ABSTRACT

Described is a computer implemented method for updating a treatment model for a patient. Also described is a corresponding computer system and computer program product.

### TECHNICAL FIELD

The present disclosure generally relates to a computer implemented method for determining a risk score for a patient. The present disclosure also relates to a corresponding computer system and computer program product.

### BACKGROUND

Over the last decades, healthcare spending has grown rapidly, and different plans have been put forward to at least slow the growth the spending. Such plans may for example focus on implementing an in comparison higher threshold for when an individual is to be given suitable treatment, still trying to keep a quality of the healthcare within a healthcare system at a desirable level.

As an alternative, a physician, a nurse or any other form of skilled therapist or medical consultant may try to provide a recommendation to the individual with the purpose of making contextual changes that are likely to have a positive health impact on the individual, thereby reducing the risk for the individual to have to seek treatment within the healthcare system.

To be able to determine when to give and when to not give treatment to the patient, some form of pre-assessment of the individual is needed.

In assessing the individual, e.g. the physician, the nurse or any person assisting the patient, use personal experience, guidelines and best practices to as objective as possible define a present state of the individual and possibly a recommended contextual change of a suggested treatment for the individual. Although e.g. the physician or therapist maintains a high knowledge base, they are human and sometimes may not be aware of recent development within the area, may not comprehend the overall status for the individual such as all relevant medical information for the individual. In addition, the currently available best practices may in some situations be to blunt to provide an individualized treatment for the individual.

Recently, digital solutions have been introduced for assisting the physician or therapist, greatly reducing subjectiveness in regards to the physician's or therapist's decision making, and at the same time allowing for an increase “resolution” in available best practices, allowing the physician or therapist to make his decisions based on a greater amount of data. Such digital solutions may also allow for all relevant medical information for the individual to be included when defining the present state of the individual.

An example of an available digital solution for recommending contextual changes is presented in US20180165418. US20180165418 specifically discloses a system that collects data that directly characterizes the health of the individual as well as contextual data pertaining to factors that might have an impact on the health of the individual. The collected factor data is used by the system to construct a vector of the characteristics that are indicative of and reflect the individual's health over time (the “health vector”). The system may also evaluate the differences in the individual's health vector as it exists at different points in time to generate a health vector change. Using the health vector and health vector change of the individual, the system determines a current health score of the individual, which characterizes the overall health of the individual (e.g., on a spectrum from very healthy to very unhealthy) at that point in time. By periodically generating health scores based on more recent health vector information, the system also constructs a trend of the individual's health changes as the individual's health score varies over time (the “health score trend”). The system compares the individual's health score trend data with data reflecting the health score trend of similarly-situated people (i.e., one or more population cohorts), and, based on that comparison and the behavior patterns of the compared cohorts, generates recommendations for actions or changes that the individual can take that are both likely to improve the individual's health as well as likely to be adopted by the individual.

However, the solution presented in US20180165418 have some general shortcomings. First of all, the solution presented in US20180165418 is blunt in the assessment of the individual, in the end resulting in that the physician/therapist may decide to bypass a possible recommendation by the digital solution to “be on the safe side” and to ensure that the individual is satisfied.

Secondly, the solution presented in US20180165418 is solely applicable to general recommendations to the individual and is not in any way focusing on actions needed to be taken when the individual has been hospitalized or needs actual treatment within the healthcare system. The solution presented in US20180165418 will thus not solve the problem of increasing healthcare spending, specifically once the individual has to be provided with actual treatment within the healthcare system.

With the above in mind, there appears to be room for further improvements of digital solutions for physicians, balancing assessment reliability and healthcare quality with the overall intention to give the individual the most suitable kind of treatment, for the individual's present health/situation.

### SUMMARY

According to an aspect of the present disclosure, the above is alleviated by a computer implemented method performed by a control unit for determining a risk score for a patient, wherein the method comprises the steps of receiving, at the control unit, a first set of individual parameters indicative of a present or a previous state of the patient, forming, using the control unit, an individual patient model based on the first set of individual parameters, determining, using the control unit, a matching level between the individual patient model and each of a plurality of different predefined generic patient models, each of the generic patient models having a predefined patient risk score, selecting, using the control unit, at least one generic patient model having a matching level above a predetermined threshold, and determining, using the control unit, the risk score for the patient based on the at least one selected generic patient model.

The overall idea with the present disclosure is to determine a risk score for the patient, where a pre-assessment of the patient is used as the main input. The risk score may in turn be used within the healthcare system for providing the patient with the most suitable treatment. In line with the present disclosure the determination of the risk score for the patient is, in comparison to prior art, not simply based on the pre-assessment of the patient but involves a process where data about the patient is matched to a plurality of different generic patient models. The different generic patient models have been formed in advance, possibly in close collaboration with experts in different fields, where the different generic patient models generally may be seen as connected to different patient behaviors and outcomes, e.g. in case of not being suitably treated. Furthermore, the different generic patient models are typically not based on knowledge about a single patient but based on general (typically anonymized) knowledge about a large population of patients and the expected (combined) outcome for those patients.

Accordingly, in line with the present disclosure, an individual model for the patient (being dependent on collected data for the patient) is matched to the plurality of different generic patient models at least one generic patient model having a matching level above a predetermined threshold is selected. Thus, instead of just determining the risk score for the patient based on a direct assessment of the patient, the present scheme ensures that the assessment of the patient is put into a “bigger picture”, by matching the specific behavior of the patient with a “cluster” of patients that have appeared/behaved in a similar manner.

By means of the present disclosure it is thus possible to rely on a general patient behavior for determining the risk score for the patient, rather than just relying on the individual patient. An advantage following by the present scheme is thus the possibility to reliably predict an expected future behavior of the patient, and how this possible behavior should be best handled to minimize complications for the patient. The matching with the different predefined generic patient models may also be seen as a way of filtering out possible variations in the individual parameters for the patient, since such variations possibly may have previously been determined to have low impact on the future for the patient.

Accordingly, the present disclosure may be targeted to ensure that the quality of treatment provided to the patient is improved while at the same time ensuring that “over treatment” is reduced, thus reducing the overall burden on the healthcare system. Furthermore, the present disclosure may be implemented in a highly flexible manner, possibly ensuring that “new” or “updated” generic patient models may be introduced along the way, taking into account newly identified “best practice”.

Within the context of the present disclosure the expression “set of individual parameters indicative of a present or a previous state of the patient” should be interpreted broadly and include any type of relevant information that has been or is collected about the patient. Such information may for example include the patient's clinical data collected over a predetermined time period (including anything from seconds/hours to over the lifetime of the patient, for example collected at different doctors' appointments and/or hospitalizations), such as including but not limited to patient vitals, number of hospitalizations, laboratory results, and prescribed medications. Further information that may be relevant for use include for example heart rate data, electrocardiograph (EKG/ECG) data, respiration rate data, patient temperature data, pulse oximetry data, and blood pressure data.

Other parameters relating to the patient is of course possible, for example including BMI, continence, incontinence, skin type visual risk areas, sex and age, malnutrition screening too (MTS), mobility, other physical conditions, a mental condition, activity, sensory perception, moisture of the body part of the patient, nutrition, friction and shear of the body part of the patient, body temperature, information relating to a previous pressure ulcer, perfusion (blood flow), diabetes, tissue perfusion and oxygenation, hygiene, hemodynamic, etc.

Preferably, the set of individual parameters may in some embodiments comprise at least one of an image and a video sequence of the patient. It is however suitable to allow the caregiver to enter other information relating to the patient, such as information relating to the parameters as listed above. The image and/or video may preferably be collected using e.g. a camera arranged in communication with the control unit, where the control unit in turn may apply an image processing scheme for extracting e.g. the above listed parameters. The image processing scheme may in some embodiments be adapted to perform a normalization with previously collected data of the patient.

Furthermore, also the expression “individual patient model” should be interpreted broadly, including in one embodiment the determination of an aggregate of the set of individual parameters for the patient. However, in another embodiment the individual patient model may be defined as a “container” for the individual parameters for the patient, e.g. defined as a string of the individual parameters, possibly organized in according to a predefined standard to improve the matching with the generic patient models.

Still further, the expression “control unit” should be interpreted broadly and may include any means for providing computing power to perform the scheme according to the present disclosure. As such, the control unit (corresponding to any means for providing processing power) may possibly be implemented within a server, within a client device (e.g. computer or mobile device), or shared therebetween as will be further discussed in the detailed description of the present disclosure.

Preferably, in one embodiment of the present disclosure the step of selecting comprises selecting the generic patient model having the highest matching level. Accordingly, one specific generic patient model may in some embodiments be pinpointed as the most relevant model and the risk scoring is as such based on this match. Such an implementation may in some embodiments be preferred, for example where it is desirable to quickly determine the risk score for the patient.

However, it may as an alternative be possible to determine the risk score based on the selection of more than one single generic patient model, such as based on a combination of at least two selected generic patient models. In such an embodiment it may be desirable to apply a weight to each of the selected generic patient models, where the weight for example may be dependent on the matching level. As is apparent, such an implementation may possible give further improvement in relation to the reliability of the determined risk score but may on the other hand possibly incur slightly more processing and thus be slightly slower as compared to when only a single generic patient model is selected.

The predetermined threshold may in some embodiments be used for ensuring that the matching at least holds a certain base level. That is, in case the matching is insufficiently low, i.e. no real match is generated when comparing the individual patient model with the plurality of different predefined generic patient models, this information may be used as an indication that the physical shall make a manual assessment of the risk score for the patient, without relying on the present scheme. That said, a low matching level may also be seen as an indicator of that the individual parameters indicative of a present or a previous state of the patient are incorrect or in other way unreliable, and that it would be suitable to collect further/new information about the patient before proceeding with the determination of the risk score.

In one embodiment of the present disclosure the method further comprises the steps of defining, using the control unit, a low, a medium and a high-risk category, and assigning, using the control unit, a risk category to the patient by comparing the determined risk score for the patient with predefined risk score ranges for the different categories. Further categories may of course be included and are within the scope of the present disclosure. Such further categories may for example include an intermediate “elevated risk category” in between the medium and a high-risk category. The use of the risk categories may in some instances be useful for allowing e.g. a caregiver to get quick information on how to act in relation to the patient, where e.g. the different categories may have been previously (e.g. in training) been assigned different actions, without having to interpret the “risk score number” (e.g. being between 0-100, or otherwise defined). Accordingly, in case the patient is determined to be in the high-risk category the caregiver may quickly act to handle the patient.

As such, in one embodiment of the present disclosure the scheme may further comprise the step of forming, using the control unit, a suggested treatment for the patient based on the selected patient risk category, wherein the suggested treatment is different for the different risk categories. That is, rather than suggesting treatment for all risk categories, the present scheme makes an exclusion to only provide suggested treatments in case it is “really” needed, possibly lowering the overall burden on the healthcare system, since only provide treatment for patients in real need will greatly reduce the overall cost for treatment. It should be understood that the risk score ranges for the different categories may be dynamic, meaning that they may change over time or with the purpose of averaging the overall cost for providing the most suitable treatment to the patient.

Preferably, the suggested treatment may comprise at least one of a “pre-treatment” for the patient or a treatment product/scheme for the patient. Within the context of the present disclosure, the expression “pre-treatment” may be any form of treatment provided to the patient before e.g. a problem has initiated. Such pre-treatment may include anything from nutrition recommendations to hygiene instructions, etc. Similarly, the expression “treatment product/scheme” should be interpreted broadly, including any form or means suitable for use in relation to active treatment of a patient, such as e.g. for treating a wound of a patient. In regards to wound product, as an example, a wound product may for example include a wound dressing, a bandage, a topical applicant, a treatment methodology in combination with a specific type of wound dressing, etc. Further, present or future, treatment product/schemes are possible and within the scope of the present disclosure.

In some embodiments the method further comprises the steps of receiving, at the control unit, a second set of individual parameters indicative of a state of the patient subsequently to receiving the suggested treatment, determining, using the control unit, a patient health progression based on the first and the second set of individual parameters, and comparing, using the control unit, the determined patient health progression with a predefined health progression being defined for the at least one selected generic patient model.

In accordance to the present disclosure is may be possible to allow a time difference between the collection of the first and the second set of individual parameters, e.g. between 1 h-90 days. The mentioned time different is however only an example, and the time difference may of course be both shorter and longer. In one embodiment it may be possible to allow the time difference to be dependent on the suggested treatment. It should furthermore be understood that more than a first and a second set of individual parameters may be used by the system, such as a third set of individual parameters, possibly allowing the time difference between when the data is collected to be fixed or variable.

Furthermore, at least some of the generic patient models may be provided with thereto related health progressions. That is, generic patient models with (or without) related treatment recommendations/suggestions may have thereto defined expectations on how as to the patient. In line with this embodiment, it may be possible to compared how the patient in fact reacted to the suggested treatment, as compared to what is expected (dependent on the selected generic patient model). The comparison may in turn be used for further develop the scheme according to the present disclosure. That is, in some embodiments it may be possible to “validate” the selected generic patient model, such as in a case where the health progression for the patient in essence corresponds to the predefined health progression for the selected generic patient model. The validation may possibly include updating the selected generic patient model with further data or minor adjustments.

However, it may be equally useful to collect and store the patient progress also in situations where the health progression for the patient deviates (sufficiently) from the predefined health progression for the selected generic patient model. In such a situation it may for example be possible to form a starting point for (or a new generic patient model), where the deviating health progression may be seen as a new situation compared to what was previously expected.

The information later information collected about the patient may not only be used for updating/adjusting/validating a generic patient model. Rather, the overall scheme according to the present disclosure may be used for allowing different institutions and/or organizations to benchmark against other. As such, it may in some embodiments be desirable to ensure that the information collected about the patient is kept strictly anonymous

Updating/adjusting the generic patient model may in one embodiment comprises applying a machine learning process. That is, rather than having a physician (or technician) forming new generic patient models, the system in itself may form such models or model iterations of already available generic patient models. For example, one generic patient model may in some situations be subdivided into two (or even further) sub-models in case further data is provided that suggests that different assessments may be made in different situations. The machine learning process may possibly be an unsupervised machine learning process, a supervised machine learning process and/or be based on a convolutional neural network (CNN) or a recurrent neural network (RNN). Further implementations are possible and within the scope of the present disclosure.

According to another aspect of the present disclosure, there is further provided a computer implemented method performed by a control unit for determining a risk score for a patient, wherein the method comprises the steps of receiving, at the control unit, a first set of individual parameters indicative of a present or a previous state of the patient, matching, using the control unit, the first set of individual parameters with a plurality of different predefined generic patient models, each of the generic patient models having a predefined patient risk score, selecting, using the control unit, at least the generic patient model best matching the individual parameters, and determining the risk score for the patient based on at least the selected generic patient model. This aspect of the present disclosure provides similar advantages as discussed above in relation to the previous aspects of the present disclosure. That said, in accordance to aspect of the present disclosure it is presented a slightly different approach where the individual parameters are directly matched with the plurality of different predefined generic patient models, without the inclusion of the individual patient model. Such an implementation may in some situations be preferred, e.g. when the type of the individual parameters is expected to be the same/similar at all instances of collection.

In accordance to a still further aspect of the present disclosure, there is provided a computer implemented method performed by a control unit for reducing a health care cost relating to a patient, the method comprising receiving, at the control unit, a first set of individual parameters indicative of a present or a previous state of the patient, forming, using the control unit, an individual patient model based on the first set of individual parameters, determining, using the control unit, a matching level between the individual patient model and each of a plurality of different predefined generic patient models, each of the generic patient models having a predefined patient risk score, selecting, using the control unit, at least one generic patient model having a matching level above a predetermined threshold, determining, using the control unit, the risk score for the patient based on the at least one selected generic patient model, defining, using the control unit, a low, a medium and a high-risk category, assigning, using the control unit, a risk category to the patient by comparing the determined risk score for the patient with predefined risk score ranges for the different categories, and suggesting, using the control unit, a treatment for the patient only if the patient has been assigned the high-risk category. Also this aspect of the present disclosure provides similar advantages as discussed above in relation to the previous aspects of the present disclosure.

Furthermore, in accordance to another aspect of the present disclosure, there is provided a computer system adapted for determining a risk score for a patient, the computer system comprising a control unit adapted to receive a first set of individual parameters indicative of a present or a previous state of the patient, form an individual patient model based on the first set of individual parameters, determine a matching level between the individual patient model and each of a plurality of different predefined generic patient models, each of the generic patient models having a predefined patient risk score, select at least one generic patient model having a matching level above a predetermined threshold, and determine the risk score for the patient based on the at least one selected generic patient model. This aspect of the present disclosure provides similar advantages as discussed above in relation to the previous aspects of the present disclosure.

In a possible embodiment of the present disclosure the computer system is a mobile electronic device, such as e.g. at least one of a “dedicated electronic device”, a mobile phone, a tablet, etc. The computer system may as an alternative be computer (e.g. laptop), for example provided with the above discussed camera for acquiring an image or video sequence of the wound of the patient. The computer system may for example be arranged to be operated by the caregiver.

In a preferred embodiment of the present disclosure the computer system comprises a graphical user interface (GUI) adapted to provide the caregiver with an instruction for acquiring the first set of parameters of the patient. The GUI may then, following the above discussed processing steps, be adapted to present the information indicative of the risk score and or the risk category.

In accordance to a still further aspect of the present disclosure there is provided a computer program product comprising a non-transitory computer readable medium having stored thereon computer program means for operating a computer system adapted for determining a risk score for a patient, the computer system comprising a control unit, wherein the computer program product comprises code for receiving, at the control unit, a first set of individual parameters indicative of a present or a previous state of the patient, code for forming, using the control unit, an individual patient model based on the first set of individual parameters, code for determining, using the control unit, a matching level between the individual patient model and each of a plurality of different predefined generic patient models, each of the generic patient models having a predefined patient risk score, code for selecting, using the control unit, at least one generic patient model having a matching level above a predetermined threshold, and code for determining, using the control unit, the risk score for the patient based on the at least one selected generic patient model. Also this aspect of the present disclosure provides similar advantages as discussed above in relation to the previous aspects of the present disclosure.

The control unit is preferably a microprocessor. Similarly, the computer readable medium may be any type of memory device, including one of a removable nonvolatile random-access memory, a hard disk drive, a floppy disk, a CD-ROM, a DVD-ROM, a USB memory, an SD memory card, or a similar computer readable medium known in the art.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1 conceptually shows a computer system according to a currently preferred embodiment of the present disclosure,

FIG. 2 discloses a possible client device comprising a graphical user interface for applying the present concept, and

FIG. 3 is a flow chart illustrating the steps of performing the method according to a currently preferred embodiment of the present disclosure.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

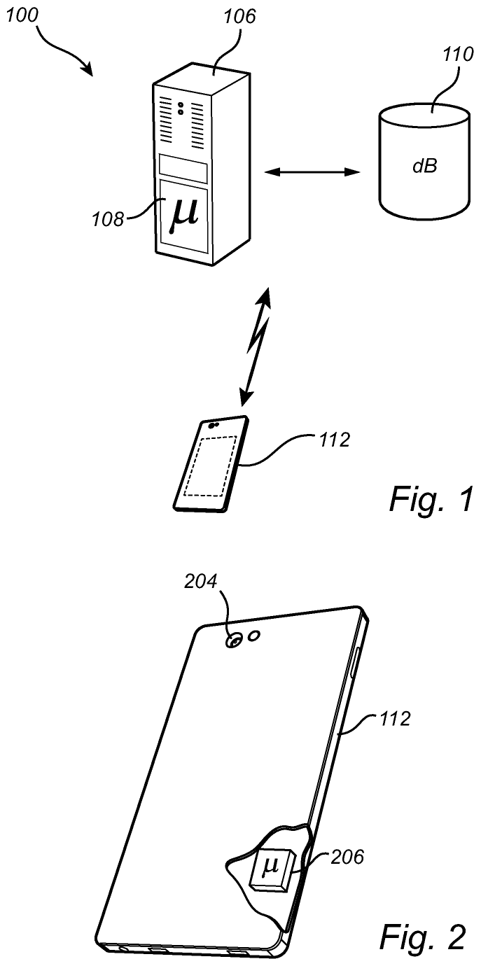

Turning now to the drawings and to FIG. 1 in particular, there is conceptually illustrated a computer system 100 adapted for determining a risk score for a patient.

The computer system 100 comprises a server 106 including some form of control unit 108 providing computing power and arranged in communication with a database 110, and a client device 112 arranged in networked communication, such as using the Internet, with the server 106. In FIG. 1, the client device 112 is operated by a caregiver (not shown); however, any user, such as for example any form of caregiver, may be allowed to operate the client device 112.

The networked communication may be wired or wireless, including for example wired connections like a building LAN, a WAN, an Ethernet network, an IP network, etc., and wireless connections like WLAN, CDMA, GSM, GPRS, 3G mobile communications, 4G mobile communications, 5G mobile communications Bluetooth, infrared, or similar.

The client device 112, as further detailed in FIG. 2 and illustrated as a mobile phone, comprises a graphical user interface (GUI) and a camera 204. The client device 112 also comprises some form of control unit 206 providing computing power. The GUI is preferably adapted to present instructions and information to e.g. the caregiver, such as for acquiring images of the patient 106 using the camera 204, for receiving further patient data inputted by the caregiver, and for displaying information in regards to the risk score for the patient and/or a risk category for the patient.

The control unit 108 as well as the control unit 206 may include a general-purpose processor, an application specific processor, a circuit containing processing components, a group of distributed processing components, a group of distributed computers configured for processing, etc. The processor may be or include any number of hardware components for conducting data or signal processing or for executing computer code stored in memory. The memory may be one or more devices for storing data and/or computer code for completing or facilitating the various methods described in the present description. The memory may include volatile memory or non-volatile memory. The memory may include database components, object code components, script components, or any other type of information structure for supporting the various activities of the present description. According to an exemplary embodiment, any distributed or local memory device may be utilized with the systems and methods of this description. According to an exemplary embodiment the memory is communicably connected to the processor (e.g., via a circuit or any other wired, wireless, or network connection) and includes computer code for executing one or more processes described herein.

Furthermore, it is in one embodiment preferred to implement the computer system 100 as a cloud-based computing system, where the server 106 is a cloud server. Thus, the computing power may be divided between a plurality of different servers (not shown), and the location of the servers must not be explicitly defined. As mentioned above, the computing power may also be distributed between the server(s) and the client device.

Advantageous following the use of a cloud-based solution is also the inherent redundancy achieved. That is, by applying a distributed approach to the server(s) as well as to the users/operators allows for an improved security as it will typically not be possible to attach (physically or computer attack) a specified operational site where e.g. a prior-art solution would hold both servers and users/operators.

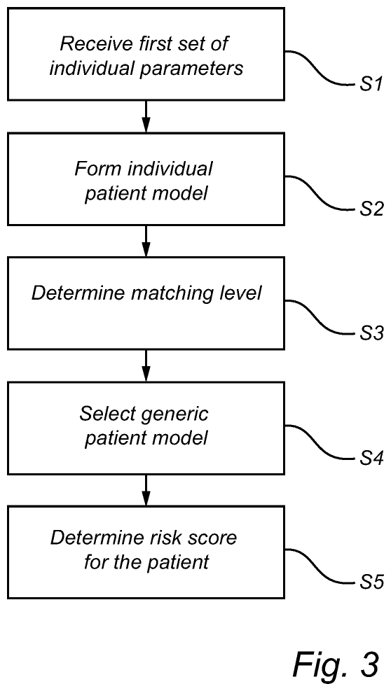

During operation of the computer system 100, with further reference to FIG. 3, the process is initiated by e.g. the caregiver providing, e.g. using the GUI of the client device 112, a first set of individual parameters indicative of a present or a previous state of the patient that is in turned received, S1, by the control unit 108 of the server and/or the control unit 206 of the client device 112.

The control unit 108/206 may subsequently form, S2, an individual patient model based on the first set of individual parameters. As mentioned above, the individual patient model may in some embodiments be a pre-assessment of the patient or may in another embodiment simply be a data string or vector holding the first set of individual parameters.

The individual patient model will then be matched, S3, with a plurality of different predefined generic patient models, where each of the generic patient models having a predefined patient risk score. The plurality of different predefined generic patient models may in some embodiments be stored with the database 110 and/or stored with a memory module comprised with the client device 112.

The matching between the individual patient model and the plurality of different predefined generic patient models will result in the determination of a matching level. The matching may in some embodiments be multi-dimensional matching, where the first set of individual parameters are matched with a multitude of different parameters relating to the plurality of different predefined generic patient models. Possibly, the first set of individual parameters may not necessarily correspond to the parameters of the plurality of different predefined generic patient models, and the plurality of different predefined generic patient models may not necessarily hold the same type of parameters. Accordingly, to find a match, it may be necessary to match parameters in multiple dimensions. The matching level should preferably take this into account and may in some embodiments include determining Euclidian distances for the different parameters.

Once the matching level has been determined, at least one generic patient model is selected, S4. That said, there is a prerequisite to only select one or a plurality of generic patient models that have a matching level above a predetermined threshold. As mentioned above, such a threshold may be dynamic and dependent on the implementation at hand. As such, the threshold may range from 0-100 (in case of the matching level having a similar range).

Following the selection of the at least one generic patient model, it is possible to determine, S5, the risk score for the patient. The risk score determination will be based on the at least one selected generic patient model but may also allow for a combination of more than a single selected generic patient model. In such an implementation the different generic patient models may have different weights, such based on their individual matching level.

The risk score may possibly be normalized between 0-100. Other ranges are of course possible and within the scope of the present disclosure. The risk score may furthermore be used for determining a risk category for the patient, possibly by allowing different ranges of the total range for the risk score, to correspond to different risk categories. In some embodiments a risk score between 0-50 may correspond to a low-risk category, 51-75 to a medium-risk category and 76-100 to a high-risk category. The provided ranges are solely for an exemplary purpose. It may be desirable to at least provide some form of treatment to patients in the high-risk category.

The present disclosure will in an efficiently manner allow for a quick and effective determination of a risk score for the patient, not just relying on data relating to the patient, but also including a matching scheme with a plurality of predefined generic patient models. Advantages following by the present scheme include the possibility to reliably predict an expected future behavior of the patient, and how this possible behavior should be best handled to minimize complications for the patient. The matching with the different predefined generic patient models may also be seen as a way of filtering out possible variations in the individual parameters for the patient, since such variations possibly may have previously been determined to have low impact on the future for the patient.

The control functionality of the present disclosure may be implemented using existing computer processors, or by a special purpose computer processor for an appropriate system, incorporated for this or another purpose, or by a hardwire system. Embodiments within the scope of the present disclosure include program products comprising machine-readable medium for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor.

Although the figures may show a sequence the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the present disclosure has been described with reference to specific exemplifying embodiments thereof, many different alterations, modifications and the like will become apparent for those skilled in the art.

In addition, variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A computer implemented method performed by a control unit for determining a risk score for a patient, wherein the method comprises the steps of:
receiving, at the control unit, a first set of individual parameters indicative of a present or a previous state of the patient,
forming, using the control unit, an individual patient model based on the first set of individual parameters,
determining, using the control unit, a matching level between the individual patient model and each of a plurality of different predefined generic patient models, each of the generic patient models having a predefined patient risk score,
selecting, using the control unit, at least one generic patient model having a matching level above a predetermined threshold, and
determining, using the control unit, the risk score for the patient based on the at least one selected generic patient model.

2. The method according to claim 1, wherein the step of selecting comprises selecting the generic patient model having the highest matching level.

3. The method according to claim 1, wherein the risk score is determined based on a combination of at least two selected generic patient models.

4. The method according to claim 3, where each of the at least two selected generic patient models each have a weight to be applied when determining the risk score.

5. The method according to claim 1, wherein the individual parameters comprise a plurality of the patient's clinical data collected over a predetermined time period.

6. The method according to claim 5, wherein the clinical data comprises at least patient vitals, number of hospitalizations, laboratory results, and prescribed medications.

7. The method according to claim 6, wherein the patient vitals comprise at least one of heart rate data, electrocardiograph (EKG/ECG) data, respiration rate data, patient temperature data, pulse oximetry data, and blood pressure data.

8. The method according to claim 1, further comprising the steps of:
defining, using the control unit, a low, a medium and a high-risk category, and
assigning, using the control unit, a risk category to the patient by comparing the determined risk score for the patient with predefined risk score ranges for the different categories.

9. The method according to claim 8, further comprising the step of:
forming, using the control unit, a suggested treatment for the patient based on the selected patient risk category,
wherein the suggested treatment is different for the different risk categories.

10. The method according to claim 9, wherein the treatment for the patient is only formed if the patient has been assigned the high-risk category.

11. The method according to claim 9, further comprising the steps of:
receiving, at the control unit, a second set of individual parameters indicative of a state of the patient subsequently to receiving the suggested treatment,
determining, using the control unit, a patient health progression based on the first and the second set of individual parameters, and
comparing, using the control unit, the determined patient health progression with a predefined health progression being defined for the at least one selected generic patient model.

12. The method according to claim 11, further comprising the step of:
updating at least one of the generic patient models based on a combination of the determined individual patient model and a result of the health progression comparison.

13. The method according to claim 12, wherein the step of updating the at least one of the generic patient model comprises applying a machine learning process.

14. The method according to claim 13, wherein the machine learning process is an unsupervised machine learning process.

15. The method according to claim 13, wherein the machine learning process is a supervised machine learning process.

16. The method according to claim 13, wherein the machine learning process is based on a convolutional neural network (CNN) or a recurrent neural network (RNN).

17. (canceled)

18. A computer implemented method performed by a control unit for reducing a health care cost relating to a patient, the method comprising:
receiving, at the control unit, a first set of individual parameters indicative of a present or a previous state of the patient,
forming, using the control unit, an individual patient model based on the first set of individual parameters,
determining, using the control unit, a matching level between the individual patient model and each of a plurality of different predefined generic patient models, each of the generic patient models having a predefined patient risk score,
selecting, using the control unit, at least one generic patient model having a matching level above a predetermined threshold,
determining, using the control unit, the risk score for the patient based on the at least one selected generic patient model,
defining, using the control unit, a low, a medium and a high-risk category,
assigning, using the control unit, a risk category to the patient by comparing the determined risk score for the patient with predefined risk score ranges for the different categories, and
suggesting, using the control unit, a treatment for the patient only if the patient has been assigned the high-risk category.

19. A computer system adapted for determining a risk score for a patient, the computer system comprising a control unit adapted to:
receive a first set of individual parameters indicative of a present or a previous state of the patient,
form an individual patient model based on the first set of individual parameters,
determine a matching level between the individual patient model and each of a plurality of different predefined generic patient models, each of the generic patient models having a predefined patient risk score,
select at least one generic patient model having a matching level above a predetermined threshold, and
determine the risk score for the patient based on the at least one selected generic patient model.

20. A computer program product comprising a non-transitory computer readable medium having stored thereon computer program means for operating a computer system adapted for determining a risk score for a patient, the computer system comprising a control unit, wherein the computer program product comprises:
code for receiving, at the control unit, a first set of individual parameters indicative of a present or a previous state of the patient,
code for forming, using the control unit, an individual patient model based on the first set of individual parameters,
code for determining, using the control unit, a matching level between the individual patient model and each of a plurality of different predefined generic patient models, each of the generic patient models having a predefined patient risk score,
code for selecting, using the control unit, at least one generic patient model having a matching level above a predetermined threshold, and
code for determining, using the control unit, the risk score for the patient based on the at least one selected generic patient model.
